(19) United States
(12) Patent Application Publication
(10) Pub. No.: US 2024/0296906 A1
(43) Pub. Date: Sep. 5, 2024

(54) Artificial selection approach for improving secondary microbial functions using a partner organism

(71) Applicant: The Trustees of Boston College, Chestnut Hill, MA (US)

(72) Inventors: Babak Momeni, Cambridge, MA (US); Marco Zaccaria, Malden, MA (US); Natalie Sandlin, Watertown, MA (US)

(73) Assignee: The Trustees of Boston College, Chestnut Hill, MA (US)

(21) Appl. No.: 18/311,385

(22) Filed: May 3, 2023

Related U.S. Application Data
(60) Provisional application No. 63/340,128, filed on May 10, 2022.

(51) Int. Cl. G16B 20/40

(52) U.S. Cl. CPC G16B 20/40

(57) ABSTRACT

A partner-assisted artificial selection (PAAS) approach, in which an assisting population acts as an intermediate to create feedback from the function of interest to the fitness of the producer. The selection for improved growth in this approach successfully leads to improved degradation performance, even in the presence of other sources of stochasticity. Successful implementation of PAAS evolves improved functions of interest such as detoxification of harmful compounds.

### CROSS-REFERENCE TO RELATED APPLICATIONS

This application claims the benefit of U.S. Provisional Application No. 63/340,128, filed on May 10, 2022, which is incorporated herein by reference in its entirety.

### STATEMENT REGARDING FEDERALLY SPONSORED RESEARCH OR DEVELOPMENT

This invention was made with government support under 2103545 awarded by the National Science Foundation and under 2021-67034-35108 awarded by USDA National Institute of Food and Agriculture. The government has certain rights in the invention.

### BACKGROUND

Microbes can have beneficial functions; for example, they are capable of breaking down complex toxic compounds. However, such functions are often adapted based on the ecological and evolutionary context of the organisms and are not necessarily optimized for applications of interest. If the function of interest has a clear fitness impact on the organism that provides that function (the “focal” organism), selection can be used to find variants with improved performance. In the absence of such fitness impact (i.e., for “secondary” functions that do not have a sizeable impact on the fitness of the focal organism), large-scale passive screening of many variants can be resorted, but such an approach is effective only when there is a high throughput assay that allows to characterize the function easily.

Thus, microbial enzymes have a broad potential to address many current needs, such as detoxification of harmful toxins and waste, but their native performance often does not match specific applications of interest. In attempting to evolve strains for a specific need, one challenge is that the functions of interest may not confer a fitness effect on the producer. As a result, a conventional selection scheme cannot be used to improve such secondary functions.

### SUMMARY

The present disclosure provides a partner-assisted artificial selection (PAAS) approach that is through artificial selection and by introducing a partner organism that senses the function of interest (e.g., is sensitive to the target toxin) provides a benefit to the focal organism. It was posited and shown computationally, that such a scheme can translate the function of interest to a fitness impact on the focal organism and allow an efficient artificial selection scheme to improve secondary functions of interest.

A wide range of applications in bioremediation was envisioned to optimize biological functions when the existing, native performance of a biological organism is not ideal for removing/deactivating toxins of interest. Beyond bioremediation, other functions can be similarly optimized as well, for example to overproduce a certain compound of interest in biotechnology or applied microbiology.

The unique advantage of this PAAS approach of the present disclosure is that it allows to implement artificial selection when the function of interest does not have a major fitness impact on the organism that provides the function. The current existing selection approach is to perform passive screening (creating many replicates and picking the best ones based on their performance), but it is less efficient and cannot be implemented when there is no high-throughput assay to probe the function.

In certain embodiments, computationally investigated the feasibility of the PAAS approach and experimentally testing the predictions are provided in the present disclosure. In other embodiments, a related, but different approach, by using biotic or abiotic hosts to select for their associated microbes with desired functions, is also provided in the present disclosure.

Other systems, methods, features, and advantages of the present disclosure can be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1. An assisting population A can generate positive feedback for D from the toxin T. The overall scheme and the specific requirements are shown on the left. On the right, a conceptual selection scheme is illustrated in which cycles of coculture (with ancestral A and evolved D) leads to improved detoxification performance of D. A droplet-based implementation was envisioned where D is clonal within each culture, but different droplets contain different variants of D.

FIGS. 2A-2B. The assisting and degrading populations can grow together and degrade the toxin of interest. The dynamics of population densities (FIG. 2A) and the toxin concentration (FIG. 2B) are shown after incorporating all interactions. In the example shown here, populations A and D are assumed to be initially at 10⁵ cells/ml and the initial toxin concentration is 10 μg/ml. All relevant parameters are listed in Table 1 below. The ImpInt model is used for this simulation.

FIGS. 3A-3B. Viability of A-D cocultures depend on the geometric mean of the initial A and D population densities. FIG. 3A. Surveying a range of initial A and D population densities shows that an increase in the initial density of one can compensate for a drop in the initial density of the other one to maintain viability. FIG. 3B. Examining the final T concentrations suggests that the geometric mean of the initial A and D population densities is the main determinant of viability and degradation performance. Final T concentrations are taken from the simulations at 72 hours. In all cases the initial toxin concentration is 10 μg/ml. All relevant parameters are listed in Table 1 below. The ImpInt model is used in these simulations.

FIGS. 4A-4C. A survey of many (n=10000) simulated instances with stochastic parameters shows that PAAS allows to select for improved detoxification as a secondary function. FIG. 4A. Scatter-plot of all instances shows a positive correlation between the detoxification rate and total cell density. The trend line is estimated based on the average total cell densities at low and high detox rates. FIG. 4B. Total cell density is also tightly linked to the effectiveness of detoxification. FIG. 4C. Comparing the distributions of the detoxification rates before selection and after selecting the top 10% instances with the highest total cell densities shows that PAAS favors improved detoxification. Final T concentrations were taken from the simulations at 46 hours. In all cases the initial toxin concentration is 10 μg/ml. All relevant parameters are listed in Table 1 below and stochastic properties are listed in Table 2 below. The ImpInt model is used in these simulations.

FIGS. 5A-5B. For optimal selection, most, but not all, of the toxin are degraded at the time of selection. FIG. 5A. Mean detox improvement (defined as the average of detoxification coefficients at the end of a round divided by its initial value) was plotted as a function of initial population densities of A and D. FIG. 5B. Mean detox improvement data in FIG. 5A was plotted as a function of the final residual T, showing an optimal performance around 1% residual T at the end of each round. For each data point, 1000 instances were sampled, with stochastic parameters listed in Table 2 below. Final T concentrations were taken from the simulations at 60 hours. In all cases the initial toxin concentration is 10 μg/ml. All relevant parameters are listed in Tables 1 and 2 below. The ImpInt model is used in these simulations.

FIGS. 6A-6C. Improvement in detox, as a function of population bottleneck. FIG. 6A. The distribution of detox improvement values is shown when different fractions of the top cases with the highest cell density were selected within a round. More stringent selections can potentially yield higher detox improvement, but at the risk of more uncertainty. FIG. 6B. Error bars are standard deviations (n=100). The grey curve is a fit into the data, with the form y=1+(y_{f}−1) x/(x+xₛ), where y_{f}=1.3 and xₛ=5. FIG. 6C. The grey curve is a linear fit into the data, y=mx, where m=2.7. Final T concentrations were taken from the simulations at 72 hours. In all cases the initial toxin concentration is 10 μg/ml. All relevant parameters are listed in Table 1 below. The ImpInt model is used in these simulations.

FIGS. 7A-7B. Stochasticity in other traits can interfere with PAAS efficiency. FIG. 7A. Correlation between total cell density and detoxification coefficient decreases as stochasticity in other traits increases. Correlation coefficient is calculated using all instances of cocultures with parameters picked from corresponding random variables. Stochasticity is defined as the ratio of σ to μ (see Table 2 below) and the same value is used for all random variables except d_{D} for which σ/μ is fixed at 0.2. Error bars are standard deviations calculated using the top 10% of instances selected based on total cell density. FIG. 7B. Detox improvement decreases with more stochasticity in other traits. Here, error bars depict bootstrap 95% confidence intervals using 100 samples of PAAS. Top 10% of the instances with the largest total population densities were selected for calculating detox improvement. All relevant parameters are similar to FIGS. 4A-4C and listed in Table 1 below. The ImpInt model is used in these simulations.

FIG. 8. Growth rate of detoxifying strains such as Rhodococcus erythropolis is minimally affected by the presence of aflatoxins, highlighting the challenge of natural selection for improved detoxification. Different concentrations of AFG₂ (dissolved in methanol) were added to basal Z culture medium (see Materials and Methods, Bacterial growth characterization) inoculated with R. erythropolis at an initial cell OD of 0.01. Cultures were allowed to grow and the initial growth rate of R. erythropolis was estimated from the increase in OD over time (as a proxy for cell density). None of the growth rates at 10, 20, or 50 μg/ml of AFG₂ were statistically different from the no-toxin control (t test, p>0.3). For comparison, the upper limit of practically relevant concentrations of AFG₂ (around 1 μg/ml) is marked by an arrow as a point of reference to show that even at much higher AFG₂ concentrations the fitness impact is minimal.

FIGS. 9A-9D. The simplified ImpInt model can adequately approximate a more mechanistic model that explicitly includes the degrading enzyme (ExpEnz). The equations behind ImpInt and ExpEnz models can be found in the Methods section (Model 1 and Model 2, respectively). The degradation coefficient in ImpInt was adjusted to match the dynamics of T offered by ExpEnz.

FIGS. 10A-10C. The simplified ImpInt model can adequately approximate a more mechanistic model that explicitly includes the resource or metabolite that mediates how population A supports population D (ExpRes). The equations behind ImpInt and ExpRes models can be found in the Methods section (Model 1 and Model 3, respectively). The degradation coefficient in ImpInt was adjusted to match the dynamics of T offered by ExpRes.

FIGS. 11A-11C. When enzyme decay rate is large, a modified implicit model that assumes degradation only by growing D cells (ImpLD) can adequately approximate the model that explicitly includes the degrading enzyme (ExpEnz). The equations behind ImpLD and ExpEnz models can be found in the Methods section (Model 4 and Model 2, respectively). The degradation coefficient in ImpLD was adjusted to match the dynamics of T offered by ExpEnz. It was noted that ImpInt no longer matches the dynamics of T from ExpEnz when the enzyme decay rate is very high.

FIGS. 12A-12B. For optimal selection, most, but not all, of the toxin are degraded at the time of selection. FIG. 12A. Detox improvement (defined as the average of detoxification coefficients at the end of a round divided by its initial value) was plotted as a function of detoxification time. Error bars show standard deviations calculated among 50 independent instances. FIG. 12B. Detox improvement data in FIG. 12A were plotted as a function of the final residual T, showing an optimal performance around 1% residual T at the end of each round. For each data point, 1000 instances were sampled, with stochastic parameters listed in Table 2 below. Initial A and D densities are 10⁵ cells/ml each. In all cases the initial toxin concentration is 10 μg/ml. All relevant parameters are listed in Tables 1 and 2 below, except K_{A}=2×10⁷ cells/ml and K_{D}=6×10⁷ cells/ml. The ImpInt model is used in these simulations. All the parameters match those in FIGS. 5A-5B.

FIGS. 13A-13F. Stochasticity in growth rates of A and D as the major contributors to the total cell density can interfere with detoxification selection. n=3000 simulated instances with stochastic parameters were surveyed to evaluate how stochasticity in parameters affects PAAS selection. FIGS. 13A-13F illustrate the scatter-plots showing that among different parameters, r_{A} and r_{D} are the most influential in determining the total cell, and can thus interfere with the ability to select for improved detoxification. Total cell density was found from simulations at 72 hours. The initial toxin concentration is 10 μg/ml. All relevant parameters are listed in Table 1 below and stochastic properties are listed in Table 2 below. The ImpInt model is used in these simulations.

FIGS. 14A-14D. Tradeoff between traits can interfere with detoxification selection. n=10000 simulated instances were surveyed to evaluate how tradeoff in parameters affects PAAS selection. Tradeoff between K_{D} and d_{D}, in the form of K_{D}=(1−φ)K_{D0}+φK_{Dm}[1−(d_{D0}−d_{Dm})/d_{Dm} was intentionally introduced. Here K_{D0} and d_{D0} are random variables with properties listed in Tables 1 and 2, d_{Dm} is the average value of d_{D0}, and φ is a free parameter that determines the strength of correlation between K_{D} and d_{D} in each instance. FIG. 14A. For φ=0.01, 0.1, and 0.9, describing examples of weak, intermediate, and strong correlation, respectively, the relation between sampled K_{D} and d_{D} values are shown. FIG. 14B. Total cell density is tightly linked to the effectiveness of detoxification in the weak tradeoff case (φ=0.01, left) but not in the strong tradeoff case (φ=0.9, right). FIG. 14C. Scatter-plot of all instances shows a positive correlation between the detoxification rate and total cell density in the weak tradeoff case (φ=0.01, left) but the correlation turns negative in the strong tradeoff case (φ=0.9, right). FIG. 14D. Comparing the distributions of the detoxification rates before selection (top, grey) and after selecting the top 10% instances with the highest total cell densities (bottom, pink) shows that PAAS favors improved detoxification in the weak tradeoff case (φ=0.01, left) but not in the strong tradeoff case (φ=0.9, right). Final T concentrations were taken from the simulations at 46 hours. In all cases the initial toxin concentration is 10 μg/ml. The ImpInt model is used in these simulations.

FIGS. 15A-15C. Coculture dynamics is insensitive to the initial ratios of A and D population densities. The population dynamics was followed in the two-dimensional space of A and D densities, starting from a range of initial A and D densities. Overall, the outcomes appeared to be largely independent of the details of the initial population ratios. FIG. 15A. With r_{A}-ρ_{T}T₀>0 and small death rates of A and D (here 0.005/hr), the trajectories of the population dynamics are independent of the initial density of A. Additionally, all cases remain viable. FIG. 15B. With r_{A}-ρ_{T}T₀>0 and at higher death rates of A and D (here 0.05/hr), lower initial densities of A may not be viable (assuming extinction when density of D reaches 0.1 cells/ml). This is because D goes extinct before A grows enough to support it. FIG. 15C. With r_{A}-ρ_{T}T₀<0, density of A declines over time and viability is only possible when the population size of D is large enough to detoxify the culture for A before A goes extinct (not shown here; see “Conditions for Viability” in Example 1). All parameters are listed in Table 1, with the exception of ρ_{T}=0.03 ml/(μg·hr) assigned in part FIG. 15C.

Additional advantages of the present disclosure are set forth in part in the description which follows, and in part could be obvious from the description, or can be learned by practice of the disclosure. The advantages of the disclosure could be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present disclosure provides a partner-assisted artificial selection (PAAS) approach, in which an assisting population acts as an intermediate to create feedback from the function of interest to the fitness of the producer. In certain embodiments, focusing on degradation of a toxin as a case example, a simplified model was used to examine how well and under what conditions such a scheme leads to improved enzymatic function. It was found that selection for improved total cell density in this scheme successfully leads to improved degradation performance, even in the presence of other sources of stochasticity; and that standard selection considerations apply in PAAS: a more restrictive bottleneck leads to stronger selection but adds uncertainty. In other embodiments, how much stochasticity in other traits can be tolerated in PAAS was also examined. These findings offer a roadmap for successful implementation of PAAS to evolve improved functions of interest such as detoxification of harmful compounds.

Many modifications and other embodiments disclosed herein can come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan could recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As could be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art could understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It is further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “Artificial selection” refers to a selection under human control, including those systems, processes, steps or combinations of steps of selecting an individual or a group of individuals based on a particular trait of interest. It is to be understood that artificial selection therefore requires a determination by man, either directly or indirectly, based on a defined selection criterion or defined selection criteria. Artificial selection systems include phenotypic selection and genotypic selection processes. As used herein, “phenotypic selection” means an artificial selection based upon one, and possibly more, phenotypes of an individual. Phenotypic selection generally comprises progeny testing wherein the estimated breeding value of an individual is determined by generating variants of the individual and determining the performance of the progeny. Variants may be produced by different methods, including, but not limited to, natural variation among progeny, induced variation during cell division, and mutagenesis.

Artificial selection steps include e.g., determining one or more of the following parameters: selection criteria and/or breeding objective(s); one or more selection indices; one or more selection targets; selection intensity; one or both sexual partners for a single mating or for multiple matings including references and/or replacements; gene or plasmid transfer; induction of mutations e.g. using chemical agents, biological agents, or ultraviolet light exposure; the number of doublings or matings that any one or more individuals will contribute to a population and the length of time that an individual will remain in a population; generation interval; breeding value; or genetic gain. Artificial selection steps can also include e.g., performing one or more steps based on a determination of one or more parameters supra and/or selecting progeny.

As used herein, the term “population” refers to a group of individuals that potentially grow together, potentially affect each other's growth environment, and potentially exchange genetic material with each other such that they contribute genetically to the next generation, including but not limited to those individuals in a microbial culture or in a breeding program. The group can be of any size e.g., a few cells to many cells and group can have any composition, consisting of one or more natural or engineered strain, species, genus, family, order, class, phylum, kingdom, or domain, etc.

As used herein, the term “assisting population” refers to a population that supports the growth of another population.

As used herein, “Selection criterion” refers to a phenotype forming the basis for a selection decision, including the presence or absence of one or more traits or combination of traits. As used herein, “trait” refers to any property of the individuals or populations that can be measured either directly or indirectly, such as, but not limited to, consumption of a particular substrate, production rate of a metabolite, presence or absence of growth in a certain environment, the degree of growth in a certain environment, etc. A trait may be observed and assessed within the populations under investigation in an artificial selection scheme or in accompanied assays set up separately. As used herein, “quantitative trait” refers to a trait that can be measured or estimated quantitatively and described as a number or a collection of numbers.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a monomer,” “a catalyst,” or “a polymer,” includes, but is not limited to, mixtures or combinations of two or more such monomers, catalysts, or polymers, and the like.

It should be noted that ratios, concentrations, amounts, and other numerical data can be expressed herein in a range format. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it could be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y‘”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

Now having described the aspects of the present disclosure, in general, the following provides details of the present disclosure. While the present disclosure is described in connection with the following details and the corresponding text and figures, there is no intent to limit the present disclosure to the descriptions. On the contrary, the intent is to cover all alternatives, modifications, and equivalents included within the spirit and scope of the present disclosure.

The following descriptions are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The vast diversity of bacterial and fungal enzymes offers potential solutions to many current challenges, including the removal of toxic compounds. Recycling complex compounds is an integrated part of the life-style for many bacteria and fungi. The same enzymes can potentially target and remove toxins that contaminate food, water, and environment. One hurdle in employing native bacterial and fungal enzymes is that the function they are adapted for may not match the degradation of toxins of interest. As a result, the degradation performance will not meet the demands for practical applications. To improve such enzymatic functions, selection for improved activity would be a clear choice, but what if enzymatic activity against such toxins is a secondary function, where toxin presence or degradation has no direct fitness impact on the bacterial or fungal cells that produce the enzyme?

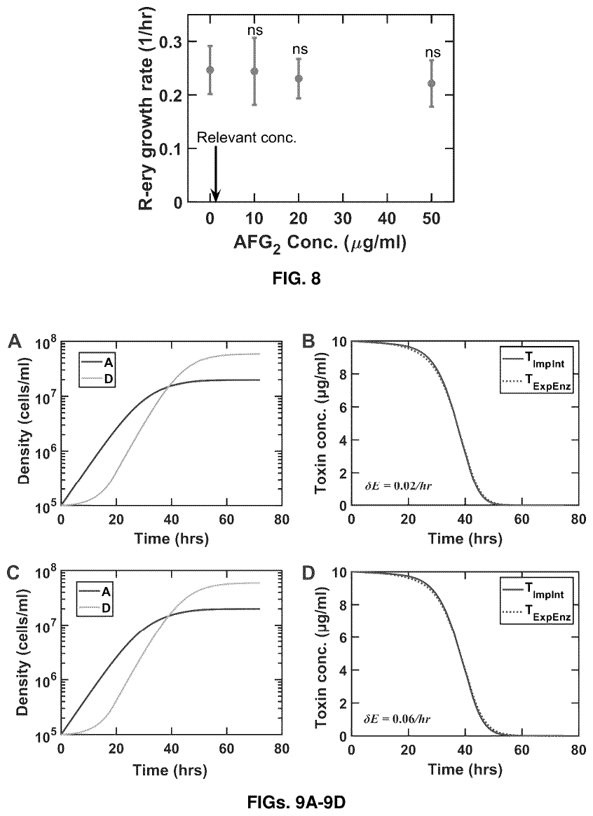

An illustrative example is the bacterial degradation of mycotoxins-fungal produced food contaminants that are toxic to consume. There are several bacteria and fungi that have already been identified to carry enzymes that degrade mycotoxins¹⁻⁶. However, at least in some cases, the presence of the toxin has little impact on the growth of bacterial cells, posing a challenge for selection. To show an example of such a situation, we have measured the growth rate of Rhodococcus erythropolis under different concentrations of aflatoxin G2 (AFG₂) in the culture (FIG. 8). Even though R. erythropolis is known to degrade aflatoxins⁷⁻⁹, AFG₂ has little positive or negative impact on its growth rate.

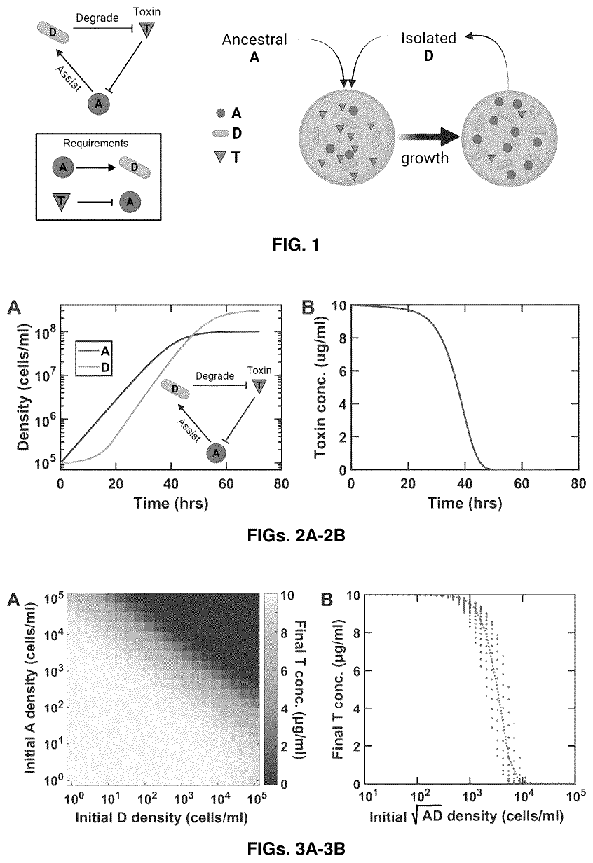

To implement a selection scheme for improving secondary microbial functions, such as detoxification of AFG₂ by Rhodococcus, the detoxification performance should be linked to the detoxifier's fitness. Adding an “assisting” partner population provides fitness feedback from the toxin to the detoxifier (FIG. 1). Community evolution has recently been revisited for its potential to improve community functions¹⁰⁻¹². Here, provided a different approach by designing a community to select for a desired microbial function. A library of variants with different quantitative traits was obtained, and the selection scheme favors variants with the best detoxification properties.

In certain embodiments, the present disclosure provides the capabilities and limitations of a partner-assisted artificial selection (PAAS) scheme to select for functions of interest that have no significant fitness impact on the cells that provide them. An assisting population that created feedback between the function of interest and the fitness of the function provider was introduced. To investigate the potentials and limits of PAAS, a system consisting of a toxin degrader was examined along with an assisting population that was sensitive to the toxin of interest and beneficial to the degrader population. As a proxy for evolutionary dynamics, how different variants fare in a single round of growth within a droplet was examined. The choice of droplets as a platform limits the interactions between different variants of the evolving toxin degrader population. Additionally, the ability to choose best-performing droplets simplifies the overall selection scheme.

It was found that selection for total cell density can lead to improved detoxification rates. This selection is most effective if it happens when detoxification is close to complete, so that there is enough discrimination between degraders with different performance. The bottleneck considerations in PAAS largely mirror the expectations in standard selection schemes. A more stringent bottleneck leads to a saturating improvement in detoxification performance, but at the cost of more uncertainty. It was also observed that too much stochasticity in other traits can mask the performance of toxin degradation and interfere with PAAS selection.

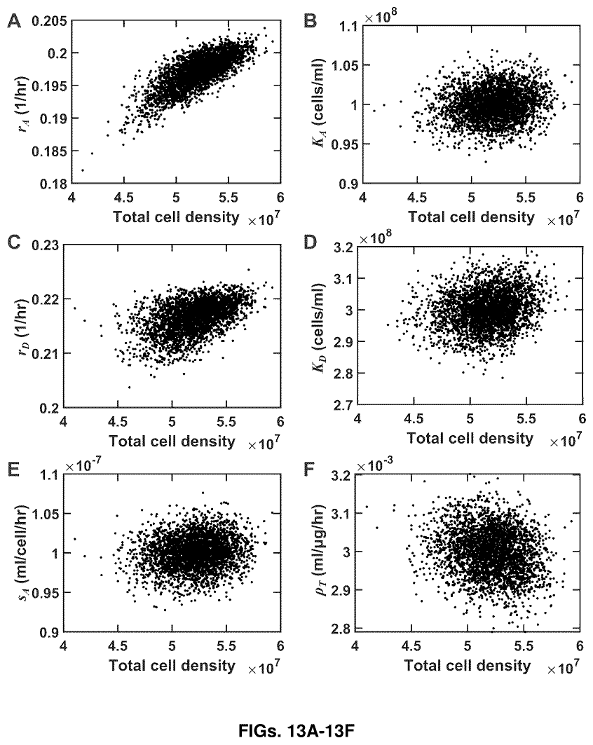

For practical implementation, the initial population sizes and the timing of selection can be used as effective design parameters. One major decision for designing an effective PAAS is the choice of bottleneck stringency; the in silico model suggests that PAAS is similar to a standard selection scheme in terms of how a more stringent bottleneck leads to stronger, but more uncertain, selection. Another major decision is the treatment of other sources of stochasticity. Among stochastic parameters that could interfere with selection, the growth rates of A and D appear to play major roles (FIGS. 13A-13F). Since the A population is reintroduced at the beginning of each round (FIG. 1, right), a pre-adaptation step to maximize its growth rate can significantly reduce the variability in this trait. In contrast, the growth rate of D, as long as it does not come at the cost of loss of degradation capabilities, could be considered a desired trait to select for.

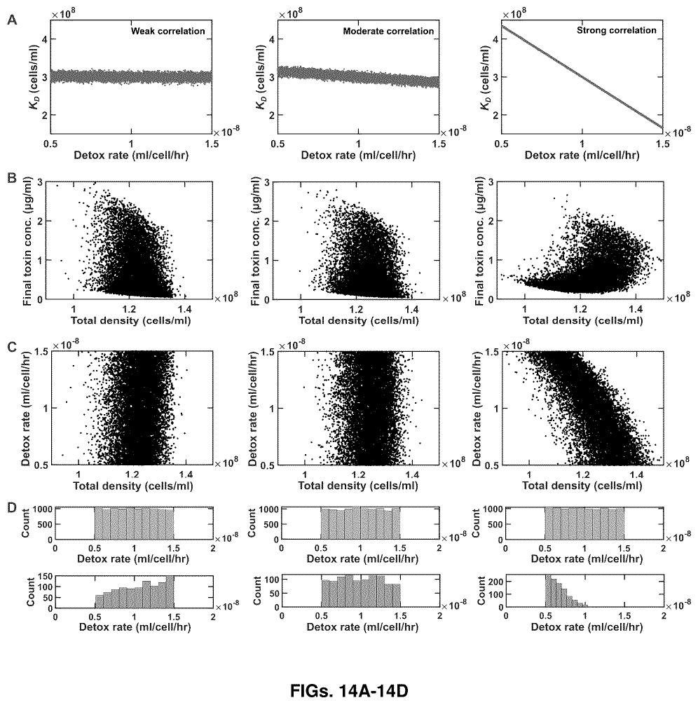

In the treatment of different traits, it was assumed that such traits are independent of each other. However, some correlation between these traits is possible, for example a positive or negative correlation between the growth rate and carrying capacity of cells¹⁶. If known, such correlations can be directly incorporated into the model for a more realistic representation of stochasticity. As an example, a tradeoff between the degradation rate (d_{D}) and the carrying capacity (K_{D}) of population D was included to account for the possibility of better degradation coming at a cost. This idea resembles the cost of providing a benefit by the associated microbes included in a model of host-microbe interactions put forward by van Vilet and Doebeli¹⁷. The results suggest that the previous conclusions hold with a weak tradeoff, but a strong tradeoff can disrupt this selection scheme (FIGS. 14A-14D). The reason is that when d_{D} and K_{D} are strongly anticorrelated, best detoxifiers no longer correspond to the highest total cell density.

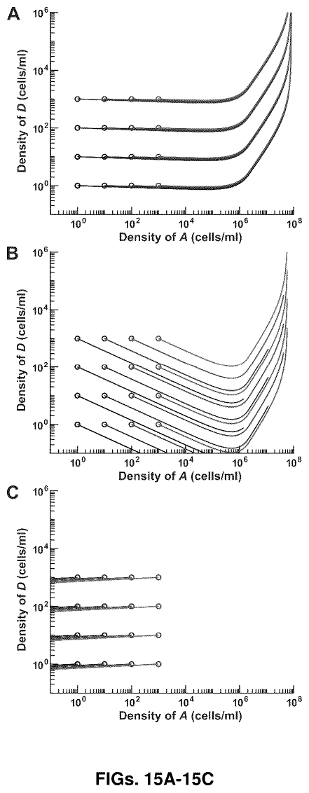

Some of the previous reports have discussed the details of community composition and its role on selection. Here, the trajectory of community dynamics appears insensitive to the details of the population composition (FIGS. 15A-15C).

One of the assumptions in the model is that there is little direct impact on A by D, be it positive or negative. This can be controlled to some extent by choosing A that satisfies this assumption or by adjusting the resources in the environment. Results are expected to be similar to the condition examined with weakly positive or negative impact on A by D. Strong positive or negative impact on A by D can change the community properties. Extreme exploitation conditions could drive A out of the community and disrupt PAAS. In contrast, strong mutualism is expected to stabilize the population dynamics 19 and lead to a more balanced performance regardless of the initial conditions.

The construction of PAAS communities is conceptually similar to the “Helper-Manufacturer” communities examined by Xie and colleagues²⁰ with one main difference: the Helper-Manufacturer system is based on commensalism, whereas the Assist-Detox system is based on mutualism. Some of the basic concepts and considerations for artificial selection, including those discussed in detail in Xie and colleagues²⁰ are shared between the two systems. However, for the specific goal of detoxification, the stronger bond between the partners in mutualism leads to stronger selection and expedites the process of finding improved detoxifiers.

Overall, the present disclosure provides that PAAS can be utilized as an additional tool to expand the power of selection to situations where the function of interest has little fitness influence on the provider of that function. It was recognized that actual implementation is likely involved adjusting the scheme to the specifics of a system of interest. The simplified model presented in the disclosure offers a baseline to build upon.

Now having described the aspects of the present disclosure, in general, the following Examples describe some additional and/or more detailed aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Materials and Methods

### Bacterial Growth Characterization

Rhodococcus erythropolis (DSM 43066) was grown from the frozen stock in glucose-yeast-malt (GYM) at 28° C. with continuous shaking (240 rpm) for 24 hrs before starting the experiments. For the growth characterization experiment, R. erythropolis was cultured in basal Z medium: KH₂PO₄ (1.5 g/L), K₂HPO₄×3H₂O (3.8 g/L), (NH₄)₂SO₄ (1.3 g/L), sodium citrate dihydrate (3.0 g/L), FeSO₄ (1.1 mg/L), glucose (4.0 g/L), 100× vitamin solution (1 mL), 1000× trace elements solution (1 mL), 1 M MgCl₂ (5 mL), 1 M CaCl₂) (1 mL), and 100× amino acid stock (10 mL). AFG₂ stock (Cayman Chemical) was dissolved in LC-MS grade methanol to the final concentration of 1 mg/mL. AFG₂ was then introduced into the growth cultures at different concentrations by further diluting the stock in methanol to keep the total methanol concentration fixed across all cases.

Final volumes of 150 μl per well were used in standard flat-bottom 96-well plates. A BioTek Synergy Mx multi-mode microplate reader was used to monitor optical density of cells at 600 nm. Reads were taken at 5 min intervals over 48 hrs. Cultures usually started at an initial OD of 0.01 and were continuously shaking between reads. Five replicates were used per condition. Only the internal wells of the 96-well plate were used for samples, and the peripheral wells of the plate were filled with sterile water to contain evaporation.

Growth rates were calculated using a Matlab code that extracted the data from text files generated by BioTek Synergy Mx. The function ‘fit_logistic’²⁰ was used to estimate the growth rates from OD readings.

### Models and Equations

There are three assumptions shared in the models. (1) The growth rate of assisting population A linearly decreases as the T concentration increases²¹. (2) The growth rate of A and its carrying capacity proportionally change at different concentrations of an inhibitor²² (3) Degradation rate of T is proportional to the density of the degrading population D. Among these assumptions, only the second assumption is necessary for the applicability of PAAS. Nonetheless, these assumptions were included to make the models more realistic, while keeping them simple.

### Model 1: Implicit Interaction Effects (ImpInt)

In this simplified model, logistic growth was assumed for the A and D populations. The toxin T is assumed to modulate both the growth rate and the carrying capacity of the population A. Growth rate and carrying capacity of population D is capped by the benefits supplied by population A.

$$\frac{dA}{dt} = ( {r}_{A} - {ρ}_{T} T ) ( 1 - \frac{A}{{K}_{A} ( 1 - {ρ}_{T} T / {r}_{A} )} ) A \qquad ( 1 )$$

$$\frac{dD}{dt} = min ( {r}_{D} , {s}_{A} A ) ( 1 - \frac{D}{{AK}_{D} / {K}_{A}} ) D \qquad ( 2 )$$

$$\frac{dT}{dt} = - {d}_{D} DT \qquad ( 3 )$$

Here D and A are the densities of A and D populations, respectively, and T is the concentration of the toxin T. In Eq. (2), the maximum growth rate is presented as min(r_{D}, S_{A}A). This choice is made to avoid the situation in which A is abundant and the growth rate of D becomes unrealistically high. The growth rate is capped at r_{D}. The same form of equations is used in the following in Model 2 and Model 4.

### Model 2: Explicit Enzyme Effect (ExpEnz)

In this model, the T-degrading enzyme (produced by D) is explicitly included. Compared to ImpInt, rather than direct degradation of T by D, D produces the enzyme E which degrades T. An explicit term for intrinsic enzyme decay was also included in the equations.

$$\frac{dA}{dt} = ( {r}_{A} - {ρ}_{T} T ) ( 1 - \frac{A}{{K}_{A} ( 1 - {ρ}_{T} T / {r}_{A} )} ) A \qquad ( 4 )$$

$$\frac{dD}{dt} = min ( {r}_{D} , {s}_{A} A ) ( 1 - \frac{D}{{AK}_{D} / {K}_{A}} ) D \qquad ( 5 )$$

$$\frac{dE}{dt} = - {η}_{D} D ( 1 - \frac{D}{γ A} ) - {δ}_{E} E \qquad ( 6 )$$

$$\frac{dT}{dt} = - {d}_{E} ET \qquad ( 7 )$$

### Model 3: Explicit Resource Effect (ExpRes)

In this model, the resource R, produced by A and supporting the growth of D, was explicitly included. A standard Monod-type growth was assumed for D on R as its main limiting resource. The consumption of R by D was also assumed to be proportional to the biomass generated by the growing D population.

$$\frac{dA}{dt} = ( {r}_{A} - {ρ}_{T} T ) ( 1 - \frac{A}{{K}_{A} ( 1 - {ρ}_{T} T / {r}_{A} )} ) A \qquad ( 8 )$$

$$\frac{dR}{dt} = {β}_{R} \frac{dA}{dt} - {α}_{D} ( \frac{R}{R + {K}_{R}} ) D \qquad ( 9 )$$

$$\frac{dD}{dt} = {r}_{D} ( \frac{R}{R + {K}_{R}} ) D \qquad ( 10 )$$

$$\frac{dT}{dt} = - {d}_{D} DT \qquad ( 11 )$$

### Model 4: Implicit Interaction Effects, Live Degradation (ImpLD)

In this modified phenomenological model, it was assumed that only growing D populations contribute to detoxification. This captures cases where the enzyme decay is large and thus detoxification stops when there is no growth and enzyme production by D cells.

$$\frac{dA}{dt} = ( {r}_{A} - {ρ}_{T} T ) ( 1 - \frac{A}{{K}_{A} ( 1 - {ρ}_{T} T / {r}_{A} )} ) A \qquad ( 12 )$$

$$\frac{dD}{dt} = min ( {r}_{D} , {s}_{A} A ) ( 1 - \frac{D}{{AK}_{D} / {K}_{A}} ) D \qquad ( 13 )$$

$$\frac{dT}{dt} = - {d}_{D} D ( 1 - \frac{D}{{AK}_{D} / {K}_{A}} ) T \qquad ( 14 )$$

### Simulations

Numerical simulations were performed using MATLAB. Source codes along with descriptions of parameters are available²¹.

Parameters and their Values

Unless otherwise stated, Table 1 lists the values of parameters used in the simulations. The order-of-magnitude of values were inferred from experimental characterization of aflatoxin G2 detoxification by Rhodococcus species.

TABLE 1
Typical parameter values for the model

| Parameter | Description | Value | |
|---|---|---|---|
| t_{f} | Total simulation time per round | 60 | hr |
| r_{A} | Maximum growth rate of population A | 0.2 | hr⁻¹ |
| r_{D} | Maximum growth rate of population D | 0.22 | hr⁻¹ |
| K_{A} | Maximum carrying capacity of population A | 10⁸ | cells/ml |
| K_{D} | Maximum carrying capacity of population D | 3 × 10⁸ | cells/ml |
| ρ_{T} | Inhibition coefficient of T against A | 0.003 | ml/(μg · hr) |
| s_{A} | Growth coefficient of A in supporting D | 10⁻⁷ | ml/(cells · hr) |
| d_{D} | Detoxification coefficient of T removal by D | 10⁻⁸ | ml/(cells · hr) |
| d_{E} | Detoxification coefficient of T removal by E | 10⁻⁸ | ml/(U · hr) |
| η_{D} | Production rate of enzyme E by D | 2.5 × 10⁻⁶ | μU/(cells · ml) |
| β_{R} | Production rate of resource R by A | 0.2 | fmole/(cells · hr) |
| K_{R} | Monod coefficient for growth of D on R | 0.2 | μM |
| α_{D} | Consumption rate of resource R by D | 0.07 | fmole/cell |
| δ_{E} | Decay rate of enzyme E | 0.02-0.5 | hr⁻¹ |

### Random Variables and Statistics

Table 2 lists the distributions used for different random variables used to include stochasticity in the simulations. For all normal random variables, the built-in random function was used in Matlab, with relevant parameters (e.g., ‘uniform’ for a uniform distribution and ‘normal’ for a normal distribution). To generate skew normal distributions for growth rates, the following transformation was used based on two independent random variables x₁ and x₂ picked from a Normal distribution [symbol] (0,1).

$${x}_{sn} = \frac{α \vert {x}_{1} \vert + {x}_{2}}{\sqrt{1 + {α}^{2}}} \qquad ( 15 )$$

Here α is the skew parameter in the distribution. The distribution is more skewed towards small (/large) values, when α is negative (/positive).

Bootstrap confidence intervals were calculated using the bootci function in Matlab, with mean as the target function.

TABLE 2
Different random variables and their distributions
in a typical artificial selection simulation

| Random | | |
|---|---|---|
| variable | Distribution | Value |
| r_{A} | Skew-normal | μ = r_{A}; σ = 0.02r_{A}; skew α = −3 |
| r_{D} | Skew-normal | μ = r_{D}; σ = 0.02r_{D}; skew α = −3 |
| K_{A} | Normal | μ = K_{A}; σ = 0.02K_{A} |
| K_{D} | Normal | μ = K_{D}; σ = 0.02K_{D} |
| ρ_{T} | Normal | μ = ρ_{T}; σ = 0.02ρ_{T} |
| s_{A} | Normal | μ = s_{A}; σ = 0.02s_{A} |
| d_{D} | Normal | μ = d_{D}; σ = 0.2d_{D} |

### Estimated Time for Detoxification

To assess viability, it needs to calculate if within the span of the observations there is a significant drop in the toxin concentration. Only weak detoxification cases are relevant for the determination of viability within the observation time t_{obs}. Additionally, it is assumed that D and A (densities of A and D populations, respectively) are away from their respective carrying capacities in these conditions, and that the growth of D is limited by the support of A. Thus, the equations are simplified to

$$\frac{d A}{dt} \approx ( {r}_{A} - {ρ}_{T} T ) A \qquad ( 16 )$$

$$\frac{d D}{dt} = {s}_{A} AD \qquad ( 17 )$$

$$\frac{d T}{dt} = - {d}_{D} D T \qquad ( 18 )$$

It was approximated (r_{A}−ρ_{T}T) as (r_{A}-ρ_{T}T₀) during this time, with the assumption that the decrease in T is small in cases that are marginally viable. Therefore,

$$A ( t ) \approx {A}_{0} exp [ ( {r}_{A} - {ρ}_{T} {T}_{0} ) t ] \approx {A}_{0} [ 1 + ( {r}_{A} - {ρ}_{T} {T}_{0} ) t ] \qquad ( 19 )$$

Then

$$\frac{d D}{d t} \approx {s}_{A} D {A}_{0} [ 1 + ( {r}_{A} - {ρ}_{T} {T}_{0} ) t ] \frac{d}{d t} ln ( D ) \approx {s}_{A} {A}_{0} [ 1 + ( {r}_{A} - {ρ}_{T} {T}_{0} ) t ] D ( t ) \approx {D}_{0} exp [ {s}_{A} {A}_{0} ( t + \frac{1}{2} ( {r}_{A} - {ρ}_{T} {T}_{0} ) {t}^{2} ) ] \qquad ( 20 )$$

Since it was assumed that changes in D are small within the observed time-scale t_{obs}, then

$$D ( t ) \approx {D}_{0} [ 1 + {s}_{A} {A}_{0} ( t + \frac{1}{2} ( {r}_{A} - {ρ}_{T} {T}_{0} ) {t}^{2} ) ] \qquad ( 21 )$$

Using this estimate, T is calculated as

$$\frac{1}{T} \frac{d T}{d t} = - {d}_{D} {D}_{0} [ 1 + {s}_{A} {A}_{0} t + \frac{1}{2} {s}_{A} {A}_{0} ( {r}_{A} - {ρ}_{T} {T}_{0} ) {t}^{2} ] \frac{d}{d t} ln ( T ) = - {d}_{D} {D}_{0} [ 1 + {s}_{A} {A}_{0} t + \frac{1}{2} {s}_{A} {A}_{0} ( {r}_{A} - {ρ}_{T} {T}_{0} ) {t}^{2} ] T ( t ) = {T}_{0} exp \{ - {d}_{D} {D}_{0} [ t + \frac{1}{2} {s}_{A} {A}_{0} {t}^{2} + \frac{1}{6} {s}_{A} {A}_{0} ( {r}_{A} - {ρ}_{T} {T}_{0} ) {t}^{3} ] \} \qquad ( 22 )$$

The threshold for the culture to be functional (i.e., at least 50% detoxification) is

$$\frac{T ( {t}_{o b s} )}{{T}_{0}} = exp \{ - {d}_{D} {D}_{0} [ {t}_{o b s} + \frac{1}{2} {s}_{A} {A}_{0} {t}_{obs}^{2} + \frac{1}{6} {s}_{A} {A}_{0} ( {r}_{A} - {ρ}_{T} {T}_{0} ) {t}_{obs}^{3} ] \} < 0.5 {d}_{D} {D}_{0} [ {t}_{o b s} + \frac{1}{2} {s}_{A} {A}_{0} {t}_{obs}^{2} + \frac{1}{6} {s}_{A} {A}_{0} ( {r}_{A} - {ρ}_{T} {T}_{0} ) {t}_{obs}^{3} ] > 0.69 \frac{1}{6} {s}_{A} {A}_{0} ( {r}_{A} - {ρ}_{T} {T}_{0} ) {t}_{obs}^{3} + \frac{1}{6} {s}_{A} {A}_{0} {t}_{obs}^{2} + {t}_{o b s} - \frac{0.69}{{d}_{D} {D}_{0}} > 0 \qquad ( 23 )$$

With (r_{A}−ρ_{T}T₀)>0 this third-degree polynomial is monotonic, with a single positive solution for t_{obs}. If (r_{A}−ρ_{T}T₀)<0, the first-order derivative of this third-degree polynomial has one positive and one negative zeros, and since the value of the function is negative at t_{obs}=0, again there will be a single positive solution for t_{obs}.

### Conditions for Viability

Starting from the equations for ImpInt,

$$\frac{d A}{d t} = ( {r}_{A} - {ρ}_{T} T ) ( 1 - \frac{A}{{K}_{A} ( 1 - {ρ}_{T} T / {r}_{A} )} ) A$$

$$\frac{d D}{d t} = min ( {r}_{D} , {s}_{A} A ) ( 1 - \frac{D}{A {K}_{D} / {K}_{A}} ) D$$

$$\frac{d T}{d t} = - {d}_{D} D T$$

Conditions that would determine the minimum requirements for viability were focused on. It was noted that if the observation time is long enough, all cultures are viable in this formulation (the fixed point has A and D at their saturation densities and T at zero). A more realistic representation is obtained if an explicit death rate (8) is added to account for population decline in the absence of growth.

$$\frac{d A}{d t} = ( {r}_{A} - {ρ}_{T} T ) ( 1 - \frac{A}{{K}_{A} ( 1 - {ρ}_{T} T / {r}_{A} )} ) A - δ A \qquad ( 24 )$$

$$\frac{d D}{d t} = min ( {r}_{D} , {s}_{A} A ) ( 1 - \frac{D}{A {K}_{D} / {K}_{A}} ) D - δ D \qquad ( 25 )$$

$$\frac{d T}{d t} = - {d}_{D} D T \qquad ( 26 )$$

The analysis was separated into three regimes (FIGS. 15A-15C):

(1) r_{A}-ρ_{T}T₀>0 and Small δ

In this regime, the A population exponentially increases from the beginning. In turn, the D population increases with an increasing rate. From Eq. (23), it was found that for viability, it is sufficient if min

$$\{ \frac{1}{6} {s}_{A} {A}_{0} ( {r}_{A} - {ρ}_{T} {T}_{0} ) {t}_{obs}^{3} , \frac{1}{6} {s}_{A} {A}_{0} {t}_{obs}^{2} , {t}_{o b s} \} > \frac{0.69}{{d}_{D} {D}_{0}} .$$

This confirms that viability is achieved if the observation time is large enough, the initial detoxification by D is fast enough, or A adequately supports the growth of D.
(2) r_{A}-ρ_{T}T₀>0 and Large δ

In this regime, the A population slowly grows but the culture is viable only if the growth can support the growth of D before it goes extinct. The time-scale for decay of D (i.e. δ) becomes critical in this case and the system is expected to be viable if A grows rapidly enough within the time span of tₑ=1/δ ln (D₀/Dₑₓₜ), where Dₑₓₜ is the extinction density for population D. This will be satisfied if s_{A}A₀ exp[(r_{A}−ρ_{T}T−δ)tₑ]>δ or in other terms when

$${s}_{A} {A}_{0} exp [ \frac{{r}_{A} - {ρ}_{T} T - δ}{δ} ln ( {D}_{0} / {D}_{ext} ) ] > δ$$

(3) r_{A}-ρ_{T}T₀<0

In this regime, the A population declines and can only be rescued if detoxification by D is rapid enough. The time-scale for decay of A is approximately 1/(ρ_{T}T₀−r_{A}+δ) and the system is expected to be viable if either ln(2)/min(r_{D}, S_{A}A)<1/(ρ_{T}T₀−r_{A}+δ) (i.e., the doubling time of D is short) or 1/d_{D}D₀<1/(ρ_{T}T₀−r_{A}+δ) (i.e. detoxification happens rapidly).

### Example 2

### Introducing an Assisting Population Generated Fitness Feedback for Degraders

The situation envisioned is when a species D is identified that can degrade a specific toxin T, but the toxin has no fitness impact, positive or negative, on D. To allow selection for improved degradation activity by D, an assisting population, A, was introduced, with two requirements (FIG. 1, left): (1) A provides a benefit to D, and (2) A is sensitive to T and gets inhibited by it. Here, it was assumed the direct interaction between A and D to be commensalism, with little direct impact on A from D. In a coculture of A and D, better degradation of T by D relieves the inhibition on A and in turn provides a positive influence on D through A. Under these conditions the interaction between A and D will transition to mutualism. The positive feedback between A and D selects for variants of D that better degrade T. In the proposed selection scheme (FIG. 1, right), in each round ancestral A is paired with evolved D from a previous round in a droplet to ensure that the evolutionary pressure is focused on D. In each round, a narrow range of D genotypes in the inoculum of each droplet grow while interacting with the ancestral A; this allows us to select best-performing variants at the end of each round to inoculate the next round of droplets (FIG. 1, right). Variation among different droplets arise from variations within the D population from previous rounds of selection as well as random mutations (either natural or induced).

### Example 3

### An Implicit Model Captures Major Aspects of Population Dynamics

To assess how well the scheme in FIG. 1 works, an implicit model was employed in which the impacts of A on D, D on T, and T on A were implicitly included as fitness contributions in a population-level model (Model 1 above, referred hereinafter as “ImpInt”). As an example, FIGS. 2A-2B show the simulated dynamics of cell populations and toxin concentration starting from a given initial condition. In this example, population A grows at a slow rate initially (under inhibition by T) until its density is high enough to support the growth of population D. Rapid growth of population D leads to a rapid decrease in toxin T concentration, and in turn, a lower inhibition of population A. In this example, within ˜48 hours the toxin is completely depleted, before populations A and D reach their saturation levels.

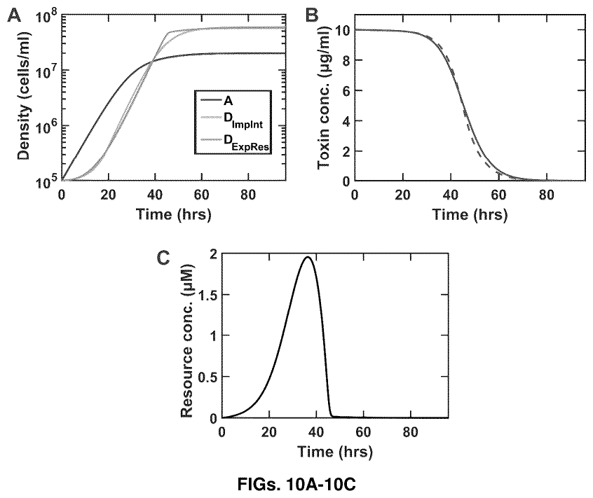

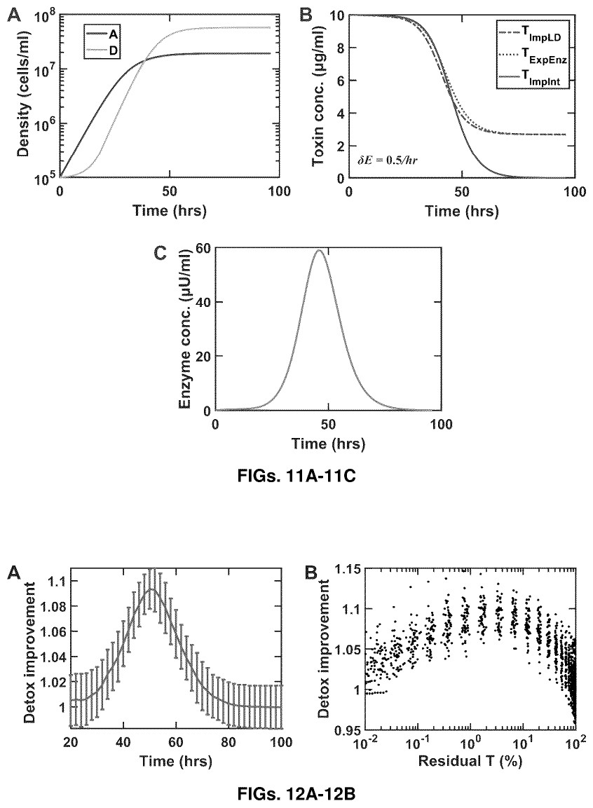

To assess whether the ImpInt model is adequate for representing this system, two more explicit models were explored: ExpEnz explicitly incorporates the T-degrading enzyme produced by D (Model 2), whereas ExpRes explicitly incorporates the resource produced by A that supports the growth of D (Model 3). It was found that ImpInt can adequately approximate the dynamics of more explicit ExpEnz and ExpRes models (FIGS. 9A-9D and 10A-10C). Given the agreement between the implicit models and the more explicit models, for simplicity ImpInt was used in the remainder of the simulations. One exception was noted when a strong enzyme decay rate was assumed in ExpEnz. For such a situation, a modified implicit model ImpLD (Model 4) had to be adopted, in which only growing D cells contribute to toxin degradation (FIGS. 11A-11C). The same approach used here can be used with ImpLD as well.

### Example 4

### Geometric Mean of a and D Population Sizes Determines Culture Viability

It was first asked under what condition a coculture of A and D is viable. The viability of a culture is defined as its ability to remove the toxin by at least 50% within the time scale of observations (e.g., 72 hours). It is expected that higher initial densities of A and D have higher propensity to be viable. A range of initial densities of A and D in the model was surveyed, which confirmed this expectation (FIG. 3A). Additionally, it appeared from this survey that a higher initial density of A or D can compensate for a lower initial density of its partner. Probing further and replotting the same data as a function of the geometric mean of the initial densities of A and D (i.e. \sqrt{A₀D₀}), it was observed that \sqrt{A₀D₀} is a good predictor of whether a coculture is viable and how well it degrades the toxin within a given time (FIG. 3B). The conditions for viability from the equations were also estimated.

### Example 5

### Despite Other Sources of Stochasticity, Selection Based on Total Cell Density Leads to Improved Detoxification

The main premise of the PAAS scheme is that effective detoxification is translated into improved overall culture growth-measured as the total cell density-a trait that can be readily selected on. To assess the efficacy of such an approach, whether variants with better detoxification rates would be selected using PAAS was computationally examined. To create a more realistic situation, it was assumed that in addition to the detoxification coefficient, other properties of the population (including their growth rates, carrying capacities, inhibition coefficient of A by T, and growth support coefficient of D by A) also varied stochastically (see Table 2). Many conditions (n=10000 instances) were then simulated with random assignments of these variables and examined the traits in the output.

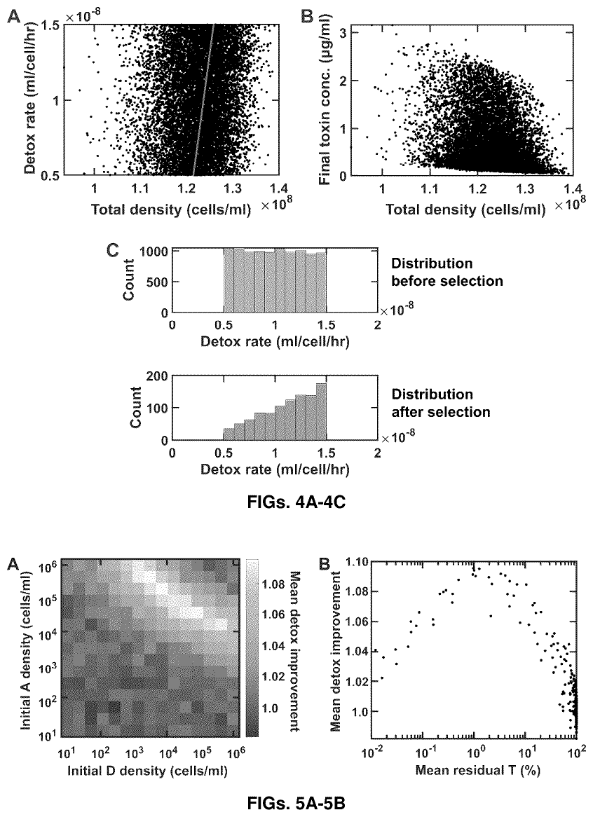

First, it was found that the detoxification rate (d_{D}) showed a positive association with overall cell density, measured in total cell density (FIG. 4A). Additionally, the overall detoxification performance was correlated with the total cell density, as expected (FIG. 4B). To examine the efficacy of selection, the distributions of the detoxification rates were compared before selection and after selecting the top 10% instances with the highest total cell densities. This selection in PAAS clearly exhibits a preference for higher detoxification rates (FIG. 4C). These results confirm the capability of PAAS to select for improved detoxifiers. Additionally, PAAS offers the advantage that cell density as the primary trait of interest is relatively easy to measure, compared to direct measurements of the toxin concentration, e.g., through fluorescence¹³, ELISA, or HPLC¹⁴⁻¹⁵.

### Example 6

### Effective Detoxification Selection is Sensitive to the Timing of Propagation

To assess the efficacy of the selection scheme, detox improvement was used as a measure of improvement in function, defined as the average detoxification rate of selected instances compared to that of initial instances. It was first assessed how the initial composition of the coculture affected detox improvement. Interestingly, the selection performance—as estimated by detox improvement—was higher in a particular range of initial densities (FIG. 5A). Further investigation revealed that this range corresponded to initial cell densities that resulted in T being mostly, but not completely, degraded. In fact, examining the data based on the residual T after 60 hours of simulated growth showed a clear trend with detox improvement being maximum around 1% residual T and dropping to lowered values when residual T was much higher or lower (FIG. 5B). This trend is intuitively expected; with too little or too much degradation, there is little information for resolving which cultures are performing well for degradation. The effect of the time between inoculation and passage was additionally examined and the results, consistent with the effect of initial density (FIGS. 5A-5B), that low, but not too low, residual T leads to the best detox improvement (FIGS. 12A-12B).

### Example 7

### Detoxification Selection Depends on the Population Bottleneck

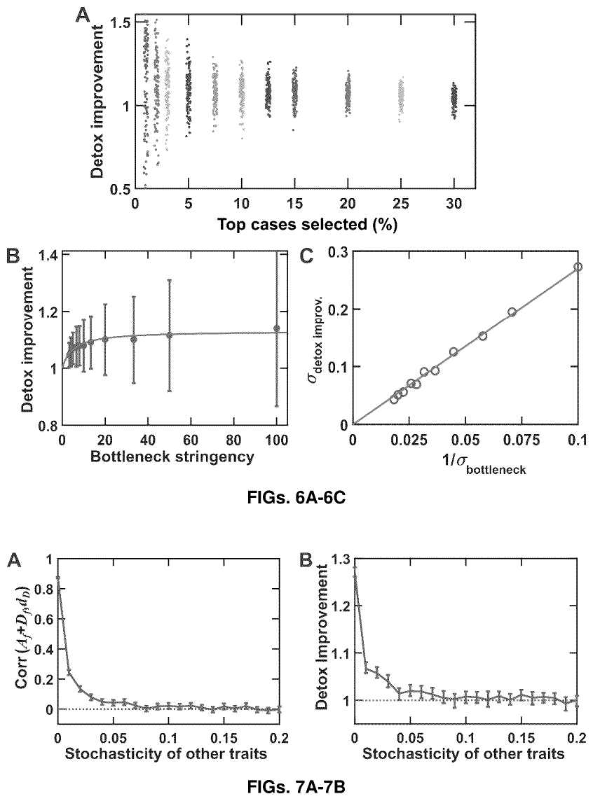

Selection is expected to depend on the size of the bottleneck. With a more stringent bottleneck (i.e., selecting more extreme cases), the expectation is to get more extreme phenotypes, but at the risk of added uncertainty of losing the best performers. It was asked if the same considerations applied to the PAAS scheme. 100 samples of the PAAS scheme were constructed, with n=100 instances of coculture (with stochastic parameters as in Table 2) in each of the samples. For each of these cases, a range of bottlenecks was enforced, from choosing the top 1% total cell density, to choosing the top 30%. The results showed that the outcome of less stringent bottlenecks was more consistent, but on average led to lower improvement (FIG. 6A). Defining bottleneck stringency as the fraction of the total number of instances to the instances selected, a saturable improvement with more stringent bottlenecks was observed (FIG. 6B). Importantly, the uncertainty in detox improvement was directly related to how stringent the bottleneck was, with σ_{bottleneck}=\sqrt{N_{bottleneck}}, and N_{bottleneck} as the size of the selected instances in the bottleneck (FIG. 6C). Overall, these follow the standard selection scheme.

### Example 8

### Stochasticity in Other Cell Traits can Disrupt Effective Selection in PAAS

Stochasticity in other parameters is one of the main factors that can potentially derail the PAAS selection scheme by muddying which cultures are the best detoxifiers. Here, how different parameters correlated with the total cell density as the main selection criterion was examiner (FIGS. 13A-13F); and how much stochasticity in other parameters can be tolerated in PAAS was also examined. For this, a range of different values of standard deviations was examined for the parameters listed in Table 2. It was found that excessive stochasticity in other traits could mask the degradation performance of the cocultures (FIGS. 7A-7B). This was evident as the correlation between detoxification coefficient and the total cell density (i.e., the criterion for selection) is lost when stochasticity in other traits is large (FIG. 7A). As a result, the selection for improved detoxification is no longer effective in such cases (FIG. 7B).

### REFERENCES

1. Abatenh E, Gizaw B, Tsegaye Z, Wassie M. The Role of Microorganisms in Bioremediation-A Review. Open Journal of Environmental Biology. 2017; 2: 038-046. doi:10.17352/ojeb.000007
2. Wang X, Qin X, Hao Z, Luo H, Yao B, Su X. Degradation of Four Major Mycotoxins by Eight Manganese Peroxidases in Presence of a Dicarboxylic Acid. Toxins 2019, Vol 11, Page 566. 2019; 11: 566. doi:10.3390/TOXINS11100566
3. Wang J, Xie Y. Review on microbial degradation of zearalenone and aflatoxins. Grain & Oil Science and Technology. 2020; 3: 117-125. doi:10.1016/J.GAOST.2020.05.002
4. Adebo O A, Njobeh P B, Gbashi S, Nwinyi O C, Mavumengwana V. Review on microbial degradation of aflatoxins. Crit Rev Food Sci Nutr. 2017; 57: 3208-3217. doi:10.1080/10408398.2015.1106440
5. Ben Taheur F, Kouidhi B, Al Qurashi Y M A, Ben Salah-Abbès J, Chaieb K. Review: Biotechnology of mycotoxins detoxification using microorganisms and enzymes. Toxicon. 2019; 160: 12-22. doi:10.1016/j.toxicon.2019.02.001
6. Sandlin N, Russell Kish D, Kim J, Zaccaria M, Momeni B. Current and emerging tools of computational biology to improve the detoxification of mycotoxins. Appl Environ Microbiol. 2021. doi:10.1128/AEM.02102-21
7. Zaccaria M, Sandlin N, Fu D, Domin M, Momeni B. Enzyme fatigue limits the detoxification of aflatoxin by Rhodococcus species. bioRxiv. 2021; 2021.08.21.457216. doi:10.1101/2021.08.21.457216
8. Alberts J, Engelbrecht Y, Steyn P, Holzapfel W, Zyl, Van W. Biological degradation of aflatoxin B1 by Rhodococcus erythropolis cultures. Int J Food Microbiol. 2006; 109: 121-126. doi:10.1016/j.ijfoodmicro.2006.01.019
9. Cserháti M, Kriszt B, Krifaton Cs, Szoboszlay S, Háhn J, Tóth Sz, et al. Mycotoxin-degradation profile of Rhodococcus strains. Int J Food Microbiol. 2013; 166: 176-185. doi:10.1016/j.ijfoodmicro.2013.06.002
10. Xie L, Yuan A E, Shou W. Simulations reveal challenges to artificial community selection and possible strategies for success. PLOS Biol. 2019; 17: e3000295. doi:10.1371/JOURNAL.PBIO.3000295
11. Xie L, Shou W. Steering ecological-evolutionary dynamics to improve artificial selection of microbial communities. Nature Communications 2021 12:1. 2021; 12: 1-15. doi:10.1038/s41467-021-26647-4
12. Sánchez Á, Vila J C C, Chang C Y, Diaz-Colunga J, Estrela S, Rebolleda-Gomez M. Directed Evolution of Microbial Communities. https://doi.org/101146/annurev-biophys-101220-072829. 2021; 50: 323-341. doi:10.1146/ANNUREV-BIOPHYS-101220-072829
13. Zaccaria M, Dawson W, Kish D R, Reverberi M, Bonaccorsi Di Patti M C, Domin M, et al. Mechanistic Insight from Full Quantum Mechanical Modeling: Laccase as a Detoxifier of Aflatoxins. bioRxiv. 2021; 2020.12.31.424992. doi:10.1101/2020.12.31.424992
14. Rodríguez Velasco M L, Calonge Delso M M, Ordónez Escudero D. ELISA and HPLC determination of the occurrence of aflatoxin M 1 in raw cow's milk. http://dx.doi.org/101080/0265203021000045208. 2010; 20: 276-280. doi:10.1080/0265203021000045208
15. Alshannaq A, Yu J H. Occurrence, toxicity, and analysis of major mycotoxins in food. Int J Environ Res Public Health. 2017; 14. doi:10.3390/ijerph14060632
16. Lipson D A. The complex relationship between microbial growth rate and yield and its implications for ecosystem processes. Front Microbiol. 2015; 6: 615. doi:10.3389/fmicb.2015.00615
17. van Vliet S, Doebeli M. The role of multilevel selection in host microbiome evolution. Proc Natl Acad Sci USA. 2019; 116: 20591-20597. doi:10.1073/PNAS.1909790116/SUPPL_FILE/PNAS.1909790116.SAPP.PDF
18. Doulcier G, Lambert A, De Monte S, Rainey P B. Eco-evolutionary dynamics of nested darwinian populations and the emergence of community-level heredity. Elife. 2020; 9: 1-40. doi:10.7554/ELIFE.53433
19. Momeni B, Brileya K A, Fields M W, Shou W, Brileya K A, Fields M W, et al. Strong inter-population cooperation leads to partner intermixing in microbial communities. Elife. 2013; 2: e00230. doi:10.7554/eLife.00230
20. Xie L, Yuan A E, Id W S. Simulations reveal challenges to artificial community selection and possible strategies for success. Siegal M L, editor. PLOS Biol. 2019; 17: e3000295. doi:10.1371/JOURNAL.PBIO.3000295
21. Niehaus L, Boland I, Liu M, Chen K, Fu D, Henckel C, et al. Microbial coexistence through chemical-mediated interactions. Nat Commun. 2019; 10: 2052. doi:10.1038/s41467-019-10062-x
22. Dedrick S. Microbial Community Structure and Function: Implications for Current and Future Respiratory Therapies. Boston College. 2021.

It should be emphasized that the embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A partner-assisted artificial selection (PAAS) method to select a desired function that has no significant fitness impact on a producer of that function, said PAAS method comprising:
a) designing a community to select a desired function; and
b) introducing an assisting population (A) that generates feedback between the desired function and the fitness impact on the producer.

2. The PAAS method of claim 1, wherein PAAS leads to improved desired function.

3. The PAAS method of claim 1, wherein the desired function is detoxification by a degrader (D) on a specific toxin T, wherein toxin T has no fitness impact on the degrader (D).

4. The PAAS method of claim 3, wherein the assisting population (A) provides a growth benefit to degrader (D) and is sensitive to and gets inhibited by toxin (T).

5. The PAAS method of claim 4, wherein a computational model with equations is employed to capture major aspects of population dynamics.

6. The PAAS method of claim 5, wherein the model (ImpInt) provides interaction effects, said model with equations is presented as follows:
$$\frac{d A}{dt} = ( {r}_{A} - {ρ}_{T} T ) ( 1 - \frac{A}{{K}_{A} ( 1 - {ρ}_{T} T / {r}_{A} )} ) A \qquad ( 1 )$$
$$\frac{d D}{dt} = min ( {r}_{D} , {s}_{A} A ) ( 1 - \frac{D}{A {K}_{D} / {K}_{A}} ) D \qquad ( 2 )$$
$$\frac{d T}{dt} = - {d}_{D} D T \qquad ( 3 )$$
Wherein D and A are the densities of a population (A) and (D), respectively, and T is the concentration of toxin (T); a maximum growth rate is presented as min(r_{D}, S_{A}A) in Eq. (2); and a growth rate is capped at r_{D}.

7. The PAAS method of claim 5, wherein the model (ExpEnz) provides enzyme effect, said model with equations is presented as follows:
$$\frac{d A}{dt} = ( {r}_{A} - {ρ}_{T} T ) ( 1 - \frac{A}{{K}_{A} ( 1 - {ρ}_{T} T / {r}_{A} )} ) A \qquad ( 4 )$$
$$\frac{d D}{dt} = min ( {r}_{D} , {s}_{A} A ) ( 1 - \frac{D}{A {K}_{D} / {K}_{A}} ) D \qquad ( 5 )$$
$$\frac{d E}{dt} = - {η}_{D} D ( 1 - \frac{D}{γ A} ) - {δ}_{E} E \qquad ( 6 )$$
$$\frac{d T}{dt} = - {d}_{E} E T \qquad ( 7 )$$
Wherein a T-degrading enzyme (produced by D) is explicitly included; and wherein D produces enzyme E which degrades T; and wherein an explicit term for intrinsic enzyme decay is also included.

8. The PAAS method of claim 5, wherein the model (ExpRes) provides resource (R) effect, said model with equations is presented as follows:
$$\frac{d A}{dt} = ( {r}_{A} - {ρ}_{T} T ) ( 1 - \frac{A}{{K}_{A} ( 1 - {ρ}_{T} T / {r}_{A} )} ) A \qquad ( 8 )$$
$$\frac{d R}{dt} = {β}_{R} \frac{d A}{dt} - {α}_{D} ( \frac{R}{R + {K}_{R}} ) D \qquad ( 9 )$$
$$\frac{d D}{dt} = {r}_{D} ( \frac{R}{R + {K}_{R}} ) D \qquad ( 10 )$$
$$\frac{d T}{dt} = - {d}_{D} D T \qquad ( 11 )$$
Wherein R, produced by A and supporting the growth of D; wherein a standard Monod-type growth for D on R as its main limiting resource is assumed; and wherein consumption of R by D is also assumed to be proportional to a biomass generated by growing D.

9. The PAAS method of claim 5, wherein the model (ImpLD) provides interaction effects and live degradation, said model with equations is presented as follows:
$$\frac{d A}{dt} = ( {r}_{A} - {ρ}_{T} T ) ( 1 - \frac{A}{{K}_{A} ( 1 - {ρ}_{T} T / {r}_{A} )} ) A \qquad ( 12 )$$
$$\frac{d D}{dt} = min ( {r}_{D} , {s}_{A} A ) ( 1 - \frac{D}{A {K}_{D} / {K}_{A}} ) D \qquad ( 13 )$$
$$\frac{d T}{dt} = - {d}_{D} D ( 1 - \frac{D}{A {K}_{D} / {K}_{A}} ) T \qquad ( 14 )$$
Wherein only growing D contributing to detoxification is assumed.

10. The PAAS method of claim 4, wherein skew normal distributions for growth rates are generated following the equation presented as follows:
$${x}_{s n} = \frac{α \vert {x}_{1} \vert + {x}_{2}}{\sqrt{1 + {α}^{2}}} \qquad ( 15 )$$
Where two independent random variables x₁ and x₂ picked from a normal distribution [symbol] (0,1); a is the skew parameter in the distribution; and wherein the distribution is more skewed towards small (/large) values, when α is negative (/positive).

11. A method to evolve improved function of interest comprising implementing the PAAS method of claim 1.

12. The method of claim 11, wherein the function of interest is detoxification of a harmful compound.
